(19) United States
(12) Patent Application Publication
(10) Pub. No.: US 2011/0276040 A1
(43) Pub. Date: Nov. 10, 2011

(54) ELASTIC STRAND FOR USE IN KINESIOLOGY TAPE

(75) Inventor: Reed Quinn, Highland, UT (US)

(73) Assignee: LUMOS, INC., Orem, UT (US)

(21) Appl. No.: 13/188,327

(22) Filed: Jul. 21, 2011

Related U.S. Application Data
(63) Continuation-in-part of application No. 12/626,355, filed on Nov. 25, 2009.
(60) Provisional application No. 61/200,400, filed on Nov. 26, 2008.

(51) Int. Cl. A61F 5/40; D02G 3/22; D02G 3/36

(52) U.S. Cl. 606/1; 428/377

(57) ABSTRACT

One example embodiment includes an elastic strand for use in kinesiology tape. The elastic strand includes an elastic core. The elastic core is configured to elongate under an external force and return to its original size after an external force is removed. The elastic strand also includes at least one outer strand, where the outer strand is wound around the elastic core.

### CROSS-REFERENCE TO RELATED APPLICATIONS

This application is a continuation-in-part of, and claims the benefit of and priority to, U.S. patent application Ser. No. 12/626,355, entitled “BODY-ADHESIVE KINESIOLOGY TAPE” filed on Nov. 25, 2009, which application is incorporated herein by reference in its entirety.

U.S. patent application Ser. No. 12/626,355 claims the benefit of and priority to U.S. Provisional Patent Application Ser. No. 61/200,400, entitled “Body-Adhesive Kinesiology Tape for Sports and Medical Use and Methods and Processes Related Thereto” filed on Nov. 26, 2008, which application is incorporated herein by reference in its entirety.

This application is related to co-pending U.S. application Ser. No. ______/______, filed on Jul. 21, 2011, entitled, “PRE-CUT STRIPS OF KINESIOLOGY TAPE” (Attorney Docket No. 10240.10), which application is incorporated herein by reference in its entirety.

This application is related to co-pending U.S. application Ser. No. ______/______, filed on Jul. 21, 2011, entitled, “MANUFACTURE OF KINESIOLOGY TAPE” (Attorney Docket No. 10240.11), which application is incorporated herein by reference in its entirety.

Co-pending U.S. application Ser. No. ______/______, filed on Jul. 21, 2011, entitled, “MANUFACTURE OF KINESIOLOGY TAPE” (Attorney Docket No. 10240.11), is a continuation-in-part of, and claims the benefit of and priority to, U.S. patent application Ser. No. 12/254,203 filed on Sep. 4, 2009, which application is incorporated herein by reference in its entirety.

### BACKGROUND OF THE INVENTION

Kinesiology tape consists of a fabric that includes elastic and non-elastic strands which is placed on human skin. Kinesiology tape is useful in therapy to reduce soreness in overused and injured muscles, tendon and joints and in rehabilitation to accelerate recovery. The tape can have a lifting effect on the skin which can reduce swelling and inflammation by improving circulation and reduce pain by taking pressure off pain receptors.

Nevertheless, there are a number of drawbacks in the current art regarding kinesiology tape. In particular, in order to provide proper support to various muscle groups or body parts, body-adhesive tapes must be applied in specific ways, which often requires that multiple strips of specific sizes and shapes be utilized. However, kinesiology tapes are generally available as a roll and the user must remove from the roll of tape the correct amount and, at times, cut the piece further, to allow the tape to properly support joints or muscles.

Body-adhesive kinesiology tapes for athletic use are required to be strong, resiliently elastic, and resistant to tearing in order to provide adequate support to a user. Such tapes cannot be easily torn into smaller pieces, but must be carefully cut into a desired size and shape. This requires that scissors be used to cut the tape into the desired shape and size. However, the scissors must be quite sharp, as the tape does not readily cut. This presents a danger to the user, as they may have to carry these scissors with them to the gym or other place of use.

Further, cutting the kinesiology tape can leave edges on the kinesiology tape with sharp corners. Since kinesiology tape is often used on or near joints, these sharp corners may continually poke or otherwise irritate the user. Moreover, the cut edges of the tape may begin to fray because of the cut. This may cause the kinesiology tape to catch on clothing and become loose while the user is participating in some physical activity. Alternatively, the user may be required to reapply the kinesiology tape during some break in the activity to ensure that the tape does not become loose or fall off.

Additionally, different joints and muscle groups may require different applications of kinesiology tape. Indeed, one joint or muscle group may need different configurations of kinesiology tape for different injuries to the joint or muscle group. Therefore, the user may need to be aware of the proper method of application as well as the type and length of kinesiology tape to apply.

Thus, those with access to professional personnel, such as personal trainers or physical therapists are able to utilize the benefits of kinesiology tapes. However, those without access to such personnel, such as a person making a casual trip to the gym, or due to other time, location or access reasons are not able to enjoy these benefits.

This can prevent casual users from receiving the support benefits from such tapes. A casual user may lack the means to cut the tape and the knowledge of the different shapes and sizes of the particular strips of tape required to support a particular body area may not be readily apparent.

Accordingly, the design of a body adhesive kinesiology tape that could be applied in multiple useful conformations without the need for custom cutting and fitting would be an improvement in the art.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes an elastic strand for use in kinesiology tape. The elastic strand includes an elastic core. The elastic core is configured to elongate under an external force and return to its original size after an external force is removed. The elastic strand also includes at least one outer strand, where the outer strand is wound around the elastic core.

Another example embodiment includes a strip of kinesiology tape. The strip of kinesiology tape includes a fabric, where the fabric includes a weave of strands. The strands include an elastic strand. The elastic strand includes an elastic core. The elastic core is configured to elongate under an external force and return to its original size after an external force is removed. The elastic strands also include at least one outer strand, where the outer strand is wound around the elastic core. The strip of kinesiology tape also includes a longitudinal cut in the fabric. The longitudinal cut: passes through at least a portion of the fabric and extends from a first edge of the fabric to a pre-determined distance from a second edge of the fabric, where the first edge is opposite the second edge. The strip of kinesiology tape further includes adhesive on a first surface of the fabric.

Another example embodiment includes a strip of kinesiology tape. The strip of kinesiology tape includes a fabric, where the fabric includes a weave of strands. The weave of strands includes a first set of strands, where each of the strands are oriented approximately parallel to one another, and a second set of strands. The second set of strands are oriented approximately parallel to one another and are oriented approximately perpendicular to the first set of strands. The second set of strands also includes an elastic strand. The elastic strand includes an elastic core. The elastic core is configured to elongate under an external force and return to its original size after an external force is removed. The elastic strands also include at least one outer strand, where the outer strand is wound around the elastic core. The fabric is approximately rectangular in shape and includes rounded corners on all exterior corners. The strip of kinesiology tape also includes a longitudinal cut in the fabric. The longitudinal cut: passes through at least a portion of the fabric and extends from a first edge of the fabric to a pre-determined distance from a second edge of the fabric, where the first edge is opposite the second edge. The longitudinal cut is approximately parallel to the second set of strands. The strip of kinesiology tape further includes adhesive on a first surface of the fabric where the adhesive is applied in a step frequency wave pattern. The step-frequency wave pattern includes a sine wave pattern with modified peaks the adhesive is configured to adhere the fabric to a human body.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an example of kinesiology tape;

FIG. 2A illustrates the adhesive pattern applied to a sheet of kinesiology tape;

FIG. 2B illustrates the adhesive pattern after the kinesiology tape has been cut into individual strips;

FIG. 3 illustrates an example of a fabric;

FIG. 4 illustrates an example of an elastic strand; and

FIG. 5 illustrates an example of an elastic strand stretched by an external force.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

In addition to providing support, body-adhesive kinesiology tapes are used by athletes for the enhancement of athletic performance and are used by athletes and non-athletes for the reduction of muscle soreness, to aid in healing from injury and in the prevention of injury. Upon application to body parts with the skin pulled taut, after returning the skin to an un-stretched position the elastic properties of body-adhesive kinesiology tapes provide an outward stretching or “lifting” force on the skin, providing enhanced fluid flow from the taped area by assisting in the opening of the lymphatic system and microcapillaries in the subcutaneous layers. Additionally, this stretching force can provide a counterbalance to muscle strain.

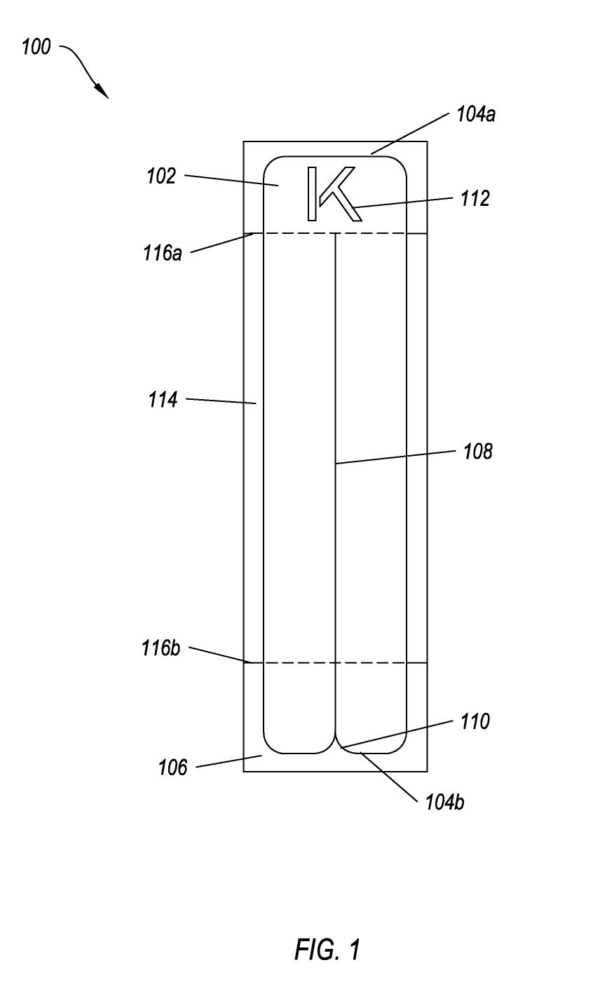

FIG. 1 illustrates an example of kinesiology tape 100. In at least one implementation, kinesiology tape 100 consists of a strip of elastic and non-elastic strands, each covered in a material which can include cotton, synthetic strands or any other desired materials, which is placed on human skin. The individual strands are woven together to produce a cloth-like tape that is able to stretch in a single direction. In particular, kinesiology tape 100 is useful in therapy to reduce soreness in overused and injured muscles, in rehabilitation to accelerate recovery and in the prevention of future injury. The kinesiology tape 100 can have a lifting effect on the skin which can reduce swelling and inflammation by improving circulation and reduce pain by taking pressure off pain receptors.

FIG. 1 shows that the kinesiology tape 100 can include a fabric 102. In at least one implementation, the fabric 102 can include any network of natural or artificial strands including textiles and cloth. In at least one implementation, the strands can include thread or yarn. For example, yarn can be produced by spinning raw wool fibers, linen, cotton, or other material on a spinning wheel to produce long strands. The fabric 102 can be formed by weaving, knitting, crocheting, knotting, or pressing fibers together, such as in felt. One of skill in the art will appreciate that the fabric 102 can include a single strand or more than one strands.

FIG. 1 shows that the fabric 102 can include an approximately rectangular shape. In particular, the kinesiology tape 100 extends in a longitudinal direction from a first end 104a to a second end 104b (collectively “ends 104”). The width and length of the kinesiology tape 100 can be changed as desired for particular applications. For example, the width can be in the range of from about 1 inch to about 3 inches, with a width of about 2 inches being used as the illustrative example discussed herein. Similarly, typical lengths of each individual strip may be from about 8 to about 12 inches, with a length of about 10 inches being used as the illustrative example discussed herein.

FIG. 1 also shows that the corner of the kinesiology tape 100 can include a rounded corner 106 on the external corners. As used herein, the term “external corner” shall include any corner on the exterior edge of the kinesiology tape 100 unless otherwise specified in the specification or claims. In at least one implementation, a rounded corner 106 can prevent fraying during application. Additionally or alternatively, a rounded corner 106 can reduce the chance of accidental detachment during use of the kinesiology tape 100. For example, a rounded corner 106 is much less likely than a square corner to snag on other materials, such as the user's clothing, that might detach the kinesiology tape 100 during use. Additionally or alternatively, a rounded corner 106 can provided more comfort to the user, as a rounded corner 106 does not have a sharp corner that can poke the user or otherwise cause discomfort.

FIG. 1 further shows that the kinesiology tape 100 can include a longitudinal cut 108. In at least one implementation, the longitudinal cut 108 can allow a user to split a portion of the kinesiology tape 100 when applying the kinesiology tape 100 to the user's body, as described below. The longitudinal cut 108 can extend from the second end 104b longitudinally into the body of the tape section progressing toward the first end 104a. In particular, the longitudinal cut 108 can extend to a termination point prior to the first end 104a, such that an uncut portion of tape is disposed at second end 104b. For example, the longitudinal cut 108 can extend from the second end 104b to a point approximately two inches from the first end 104a.

FIG. 1 also shows that the kinesiology tape 100 can include a backing material 110 disposed underneath the kinesiology tape 100. In at least one implementation, the backing material 110 is releasably attached to the kinesiology tape 100 by an adhesive layer, as described below. In particular, the backing material 110 can include paper or any other material suitable for protecting the adhesive on the kinesiology tape 100 from drying before use. For example, the backing material 110 can include a waxed paper which protects the adhesive from being removed or drying.

In at least one implementation, the backing material 110 may be formed as a continuous piece across its width, lacking a longitudinal cut which corresponds to the longitudinal cut 108 of the kinesiology tape 100. In particular, the lack of a longitudinal cut in the backing material 110 can allow the kinesiology tape 100 to be removed from the backing material 110 as one piece, without splitting the kinesiology tape down the longitudinal cut 108. This can allow the user to separate the two strips formed by longitudinal cut 108 or to leave the two strips formed by longitudinal cut 108 adjacent to one another, depending on the intended placement. In at least one implementation, the backing material 110 can be placed on the kinesiology tape 100 before longitudinal cut 108 is formed in the kinesiology tape 100. For example, longitudinal cut 108 may be formed by die cutting through the kinesiology tape 100 to the level of the backing material 110 to result in the described structures.

FIG. 1 shows that the backing material 110 can include a first perforation 112a and a second perforation 112b (collectively “perforations 112”). For example, the perforations 112 may be disposed at points about two inches from the first and second ends 104a and 104b respectively. In at least one implementation, the perforations 112 can facilitate the tearing of the backing material 110 along the perforations 112. In particular, perforations 108 can allow a portion of the backing material 110 to be removed from the kinesiology tape 100 while other portions of the backing material 110 remain on the kinesiology tape 100. This can facilitate placement of the kinesiology tape 100 by allowing the user to only work with desired sections of the kinesiology tape 100.

In at least one implementation, the backing material 110 can be placed on the kinesiology tape 100 before perforations 112 are formed in the backing material 110. For example, perforations 112 may be formed by die cutting through the backing material 110 at or near the level of the kinesiology tape 100 to result in the described structures. Additionally or alternatively, the perforations can be formed in the backing material prior to the placement of the kinesiology tape 100 on the backing material 110.

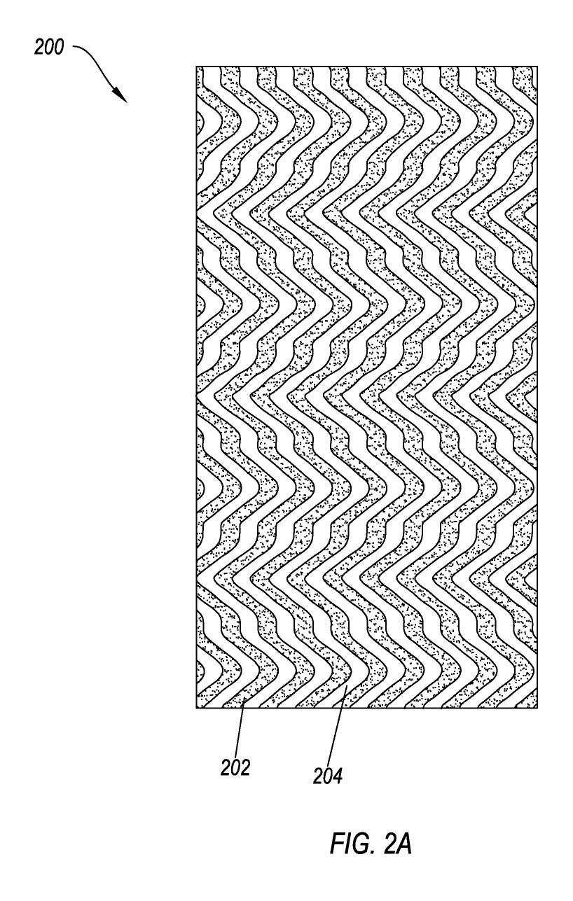

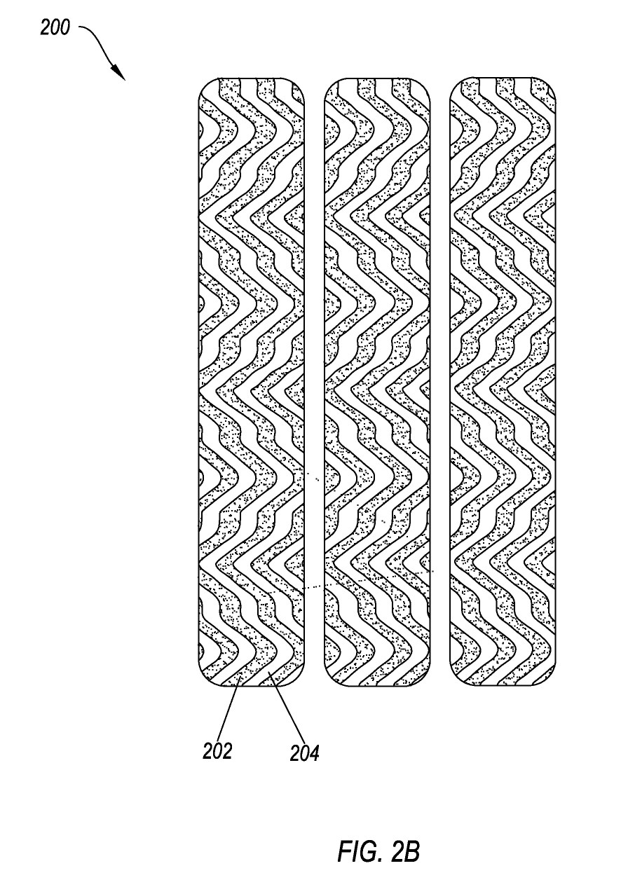

FIGS. 2A and 2B illustrate an adhesive pattern 200 that can be applied to kinesiology tape. FIG. 2A illustrates the adhesive pattern 200 applied to a sheet of kinesiology tape. FIG. 2B illustrates the adhesive pattern 200 after the kinesiology tape has been cut into individual strips. Although described herein as deposited on kinesiology tape, one of skill in the art will understand that the adhesive can be applied to a backing material with kinesiology tape later applied to the backing material or through some other method.

In at least one implementation, the adhesive can include any adhesive which will allow the kinesiology tape to adhere to the skin of a user without irritating the user's skin. In at least one implementation, the adhesive can allow the kinesiology tape to adhere to the user's skin without irritating the user's skin. In particular, the main ingredient can include a single compound or a mixture of compounds. For example, the main ingredient can include polyacrylate. Additionally or alternatively, the adhesive can include a solvent which is configured to evaporate or break down after application of the adhesive, leaving the main ingredient behind. For example the adhesive can include about 50% of the main ingredient with the rest of the adhesive comprising solvent. In at least one implementation, the solvent can include ethyl acetate.

Additionally or alternatively, the adhesive can include pressure-sensitive adhesive. Pressure sensitive adhesive is adhesive which forms a bond when pressure is applied. I.e., no solvent, water, activator chemicals, heat or other activating agent is needed to activate the adhesive. In at least one implementation, the degree of bonding is influenced by the amount of pressure which is used to apply the adhesive to the surface of the backing material. I.e., pressure applied to the backing material and kinesiology tape in combination after the application of the adhesive can be used to activate the adhesive.

FIGS. 2A and 2B show that the adhesive pattern 200 can include a step frequency pattern. In at least one implementation, a step-frequency pattern can include the adhesive applied in a modified sine wave pattern. For example, the adhesive pattern 200 includes a wave with the upper peaks (as shown in FIG. 2) modified to include a higher amplitude that includes a sharper peak. Additionally, the adhesive pattern 200 includes lower peaks (as shown in FIG. 2) modified to include a higher absolute amplitude, i.e., a higher amplitude relative to the baseline of the sine wave. In at least one implementation, the adhesive pattern 200 can provide greater adhesion for the kinesiology tape. For example, the adhesive pattern 200 can provide adhesion even with lateral movement of the kinesiology tape relative to the user's skin. In particular, the adhesive pattern 200 can provide resistance to lateral movement of the kinesiology tape in any direction on the user's skin. In at least one implementation, such resistance can allow the kinesiology tape to better provide benefits to the user during use of the kinesiology tape, as described above.

In at least one implementation, the adhesive pattern 200 can be produced using an erratic cam. Additional information regarding the use of an erratic cam to produce an adhesive pattern is provided in co-pending U.S. patent application Ser. No. ______/______, entitled “MANUFACTURE OF KINESIOLOGY TAPE,” filed Jul. 21, 2011, previously referenced.

FIGS. 2A and 2B also show that the adhesive pattern includes a series of adhesive lines 202 interrupted by gaps 204. In at least one implementation, the gaps 204 can allow the kinesiology tape to breathe. That is, the gaps 204 can allow air to reach the skin of the user. Additionally or alternatively, the gaps 204 can allow sweat from the users skin to be wicked away from the skin by the kinesiology tape. Removing sweat from the user's skin can prevent the sweat from adversely affecting the adhesion of the kinesiology tape to the user's skin.

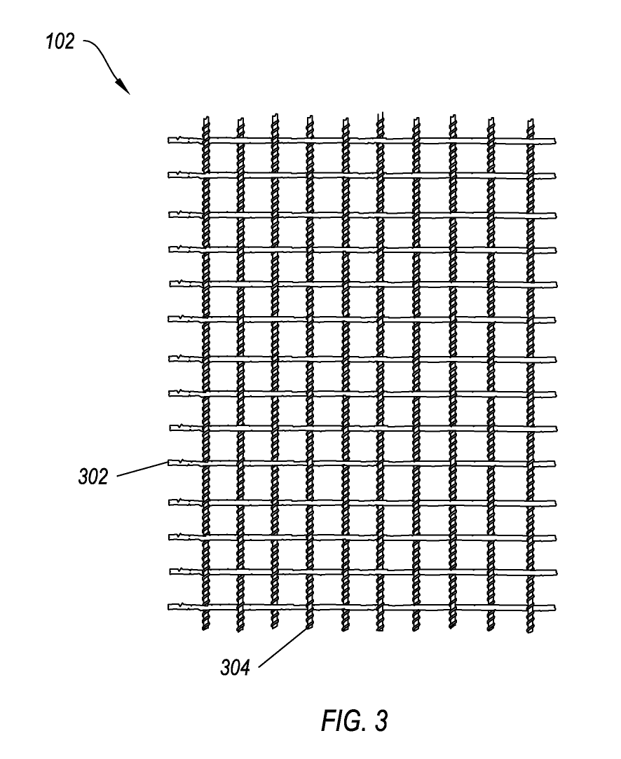

FIG. 3 illustrates an example of a fabric 102. In at least one implementation, the fabric 102 can be used as part of kinesiology tape. In particular, the fabric 102 can be applied to a user's body. The fabric 102 can provide therapeutic benefits to the user, as described above. One of skill in the art will also appreciate that the fabric 102 is only one example of fabric 102 and should not be seen as limiting of the invention.

FIG. 3 shows that the fabric 102 can include a first set of strands 302. In at least one implementation, the first set of strands 302 is a class of materials that are continuous filaments or are in discrete elongated pieces. In particular, strands 302 can be spun into filaments, string or rope, used as a component of composite materials, or matted into sheets to make products such as paper or felt. Additionally or alternatively, strands 302 can include yarn, thread, string, filaments, twine, cord, or any other material that can be used to form a fabric or cloth. In particular, yarn is a long continuous length of interlocked fibers, suitable for use in the production of textiles, sewing, crocheting, knitting, weaving, embroidery and rope making. Additionally or alternatively, thread is a type of yarn intended for sewing by hand or machine. Yarn and/or thread may be finished with wax or other lubricants to withstand the stresses involved in sewing or otherwise manufacturing the fabric 102.

Natural fibers can include animal fibers such as alpaca, angora, bison down, camel hair, cashmere, catgut, chiengora, guanaco, llama, mohair, pashmina, qiviut, rabbit, silk, sinew, spider silk, wool, vicuña, and yak; vegetable fibers such as aback bamboo, coir, cotton, flax, hemp, jute, kapok, kenaf, piña, raffia palm, ramie, sisal, and wood; mineral fibers such as asbestos, basalt, mineral wool, and glass wool; and cellulose fibers such as acetate, art silk, bamboo, lyocell (tencel), modal, and rayon (aka viscose silk); or any other natural fiber. Synthetic fibers can include acrylic, aramid (twaron, kevlar, technora, and nomex), carbon (tenax), derclon, microfiber, modacrylic, nylon, olefin, polyester, polyethylene (dyneema, spectra), spandex, vinalon, zylon, or any other synthetic fiber. One of skill in the art will appreciate that fibers 302 can include any natural or synthetic fibers, or any combination thereof, without restriction and without limitation unless otherwise indicated in the claims.

FIG. 3 also shows that the fabric 102 can include a second set of strands 304. In at least one implementation, the second set of strands 304 can be elastic. In particular, the second set of strands 304 can be capable of stretching and becoming longer than the native length of the strands 304 when an external force is applied parallel to the second set of strands 304. When the external force is removed, the second set of strands 304 returns, or attempts to return, to its original length, as described below.

FIG. 3 further shows that the first set of strands 302 and the second set of strands 304 can be used to form a grid. In particular, the first set of strands 302 can be oriented in a “horizontal” direction; that is, the first set of strands 302 can be oriented parallel to one another. One of skill in the art will appreciate that horizontal orientation refers to the orientation shown in FIG. 3 and is not used to limit or restrict the scope of the claims unless otherwise indicated in the claims. Further, the first set of strands 302 can be spaced equidistant from one another or in a repeating pattern. For example, a first strand 302a might be relatively close to a second strand 302a, which is, in turn, relatively farther from a third strand 302a, with the pattern repeating throughout the fabric 102. One of skill in the art will appreciate that the spacing of the first set of strands 302 can be any distance from one another without restriction and without limitation unless otherwise indicated in the claims.

FIG. 3 additionally shows that the second set of strands 304 can be oriented in a “vertical” direction; that is, the second set of strands 304 can be oriented parallel to one another. One of skill in the art will appreciate that vertical orientation refers to the orientation shown in FIG. 3 and is not used to limit or restrict the scope of the claims unless otherwise indicated in the claims. Further, the second set of strands 304 can be spaced equidistant from one another or in a repeating pattern. For example, a first strand 302b might be relatively close to a second strand 302b, which is, in turn, relatively farther from a third strand 302b, with the pattern repeating throughout the fabric 102. One of skill in the art will appreciate that the spacing of the second set of strands 304 can be any distance from one another without restriction and without limitation unless otherwise indicated in the claims.

In at least one implementation, the first set of strands 302 and the second set of strands 304 can be woven together. In particular, weaving is the textile art in which a first set of strands 302 and a second set of strands 304, called the warp and the filling or weft (older woof), respectively, are interlaced with each other to form a fabric or cloth. In particular, the first set of strands 302 can be held in place while the second set of strands 304 are woven through them or vice versa. The manner in which the first set of strands 302 and the second set of strands 304 interlace with each other is known as the weave. In particular, the weave can include plain weave, satin weave, and twill or any other type of weave.

In at least one implementation, plain weave can include the first set of strands 302 and second set of strands 304 aligned so they form a simple criss-cross pattern. Each of the strands in the second set of strands 304 crosses the first set of strands 302 by going over one, then under the next, and so on. The next strand in the second set of strands 304 goes under the first set of strands 302 that its neighbor went over, and vice versa. The satin weave is characterized by four or more strands in the second set of strands 304 floating over a strand in the first set of strands 304 or vice versa, four or more strands in the first set of strands 302 floating over a single strand in the second set of strands 304. Twill is a type of fabric woven with a pattern of diagonal parallel ribs. It is made by passing a strand in the second set of strands 304 over one or more strands in the first set of strands 302 and then under two or more strands in the first set of strands 302 and so on, with a “step” or offset between rows to create the characteristic diagonal pattern.

In at least one implementation, the fabric 102 can include 60-66 non-elastic strands 302 per linear inch woven when no force is applied with 60-66 elastic strands 304 per linear inch when no force is applied. I.e., one square inch of the fabric 102 can include 60-66 non-elastic strands 302 and can include 60-66 elastic strands 304. In contrast, when the fabric is made into tape, laminated with adhesive and applied to the backer paper the fabric will contain 30-35 non-elastic strands per inch and 40-45 elastic strands per inch. One of skill in the art will appreciate that more strands per inch can indicate a tighter weave, finer strands or some combination thereof. For example, the non-elastic strands 302 can include 250 D 60 F or 120 D 24 F. D represents a denier, where denier is a unit of measure for the linear mass density of strands. It is defined as the mass in grams per 9,000 meters. F represents the number of fibers used to create the strand 302. Thus, a 250 D 60 F strand 302 includes 60 fibers and 9,000 meters of a single strand containing 60 fibers will weigh 250 g. In contrast, the elastic strands 304 are made up of several components, with the specifications described below.

In at least one implementation, the second set of strands 304 can allow the fabric 102 to be uni-elastic. That is, the fabric 102 can stretch in only a single direction, rather than all directions equally. In particular, the fabric 102 can stretch in the direction parallel to the second set of strands 304 but not in the direction parallel to the first set of strands 302. This can allow the fabric 102 to confer the benefits of kinesiology tape, described above.

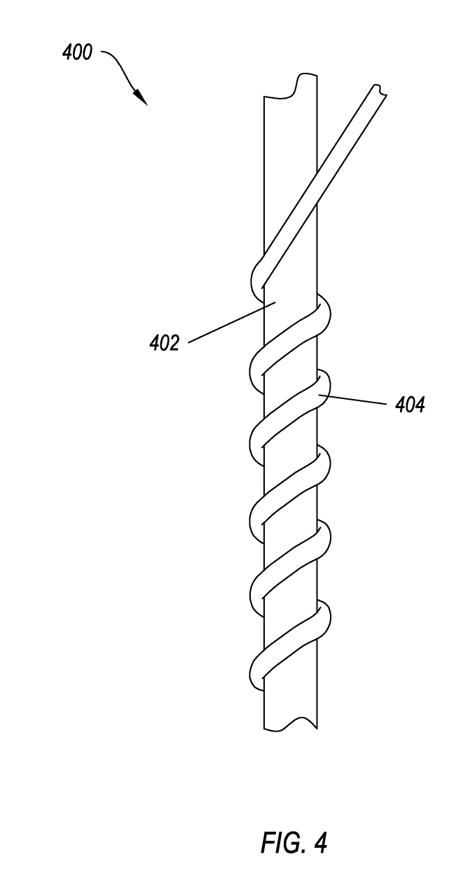

FIG. 4 illustrates an example of an elastic strand 400. In at least one implementation, the elastic strand 400 can increase in length under external forces. In particular, the elastic strand 400 can undergo reversible deformation. I.e., once the applied forces are removed, the elastic strand 400 returns to its original shape and length. One of skill in the art will appreciate that if a portion of the applied forces are removed, the elastic strand 400 can partially return to its original length and size. I.e., the user can apply enough force to stretch the elastic strand 400 to any length between its original size and 100% of its maximum size.

FIG. 4 shows that the elastic strand 400 can include an elastic core 402. In at least one implementation, the elastic core 402 can stretch laterally. I.e., the elastic core 402 can lengthen under an external force. The elastic core 402 can include any material which is capable of elastic deformation. For example, the elastic core 402 can be made of urethane or any other elastic material. The elastic core can be between 40 D and 70 D.

FIG. 4 also shows that the elastic strand 400 can include an outer strand 404. In at least one implementation, the outer strand 404 can be wrapped around the elastic core 402. In particular, the outer strand 404 can form a spiral which surrounds the elastic core 402. For example, the outer strand 404 can be wrapped around the elastic core 402 between 85 and 120 times per inch. One of skill in the art will appreciate that more than one outer strand 404 can be wrapped around the elastic core 402, forming a sheath around the elastic core 402. For example, between 15 and 30 outer strands 404 can be used to create a sheath. In particular, between 20 and 25 outer strands 404 can be used to create the sheath.

In at least one implementation, the elastic strand 400 can be described as 21 S+105 D where 21 S describes the sheath of spiral wrapped non-elastic outer strands 404 and 105 D describes the single inner elastic core 404. S represents the weight of the outer strand 404 and is a unit of measure such that 1 S would be a strand that is 840 yards long and weighs 1 pound. E.g. 21 S would mean that a 1 pound outer strand 404 would be 21×840 yards long. The higher the S value the thinner the outer strand 404. For example, the elastic strand 400 can include multiple outer strands 404 of different weights and thicknesses ranging from 12 S to 40 S. Multiple outer strands 404 can strengthen the elastic strand 400. I.e., multiple outer strands 404 can provide additional tensile strength to the elastic strand 400, preventing tearing of the elastic strand 400 when an external force is applied attempting to stretching the elastic strand 400 beyond its original length. As used in the specification and the claims, the term approximately shall mean that the value is within 10% of the stated value, unless otherwise specified.

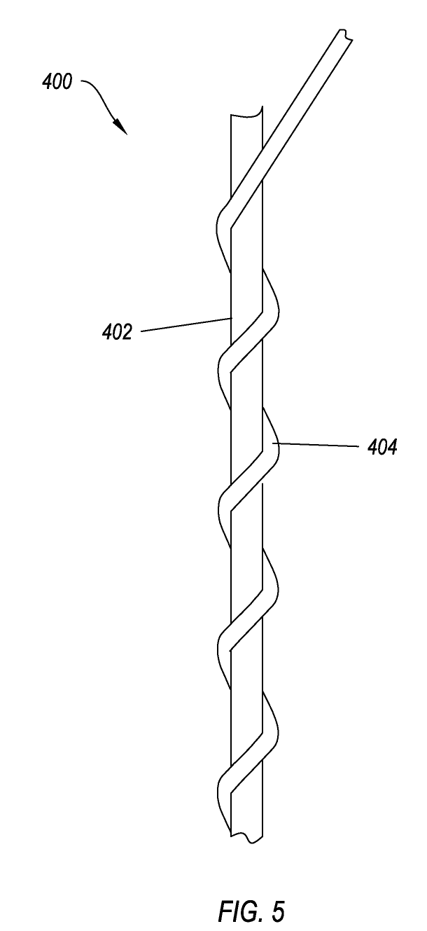

FIG. 5 illustrates an example of an elastic strand 400 stretched by an external force. In at least one implementation, the elastic strand 400 is allowed to stretch to a predetermined length. I.e., the elastic strand 400 reaches a maximum length and then resists further stretching. For example, the elastic strand 400 can be stretched between 120% and 160% of its original length. I.e., the elastic strand 400 can increase in length, under an external force, by between 20% and 60%. E.g., the elastic strand can be stretched to approximately 140% of its original length.

FIG. 5 shows that under the external force, the elastic core 402 has lengthened. In at least one implementation, as the elastic core 402 is lengthened, the diameter of the elastic core 402 is reduced. I.e., an increase in length of the elastic core 402 leads to a decrease in diameter.

Although the elastic core 402 can stretch to many times its original length, the maximum stretch is limited by the stretch of the non-elastic outer strand 404. FIG. 5 shows that under the external force, the spiral created by the outer strand 404 lengthens. As the spiral lengthens, the diameter of the spiral decreases. In at least one implementation, the decrease in the diameter of the spiral created by the outer strand 404 decrease more rapidly than the decrease in the diameter of the elastic core 402. I.e., the lengthening of the spiral created by the outer strand 404 is restricted by the more rapid decrease in the diameter of the spiral created by the outer strand 404 than the decrease in the diameter of the elastic core 402. This restricts the maximum length to which the elastic strand 400 can be stretched.

Additionally or alternatively, the length of the outer strand 402 can increase more rapidly than the length of the elastic core 402. I.e., the spiral created by the outer strand 402 increase faster than the length of the elastic core 402. As the elastic strand 400 increases in length, eventually the length of the outer strand 402 overtakes the length of the elastic core 402, preventing further lengthening of the elastic strand 400.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An elastic strand for use in kinesiology tape, the elastic strand comprising:
an elastic core, wherein the elastic core is configured to:
elongate under an external force; and
return to its original size after an external force is removed; and
at least one outer strand, wherein the outer strand is wound around the elastic core.

2. The elastic strand according to claim 1, wherein the elastic core includes urethane.

3. The elastic strand according to claim 1, wherein the outer strand is wrapped around the elastic core between 85 times per inch and 120 times per inch.

4. The elastic strand according to claim 1, wherein the elastic core is approximately round.

5. The elastic strand according to claim 4, wherein the inner diameter of the wound outer strand shrinks at a greater rate than the diameter of the elastic core if the strand is stretched lengthwise.

6. The elastic strand according to claim 5, wherein the elongation of the elastic strand is retarded when the inner diameter of the wound outer strand shrinks to the size of the outer diameter of the elastic core.

7. The elastic strand according to claim 1, wherein the elongation of the elastic strand is retarded when the elastic strand has been stretched to between 120% and 160% of its original length.

8. The elastic strand according to claim 7, wherein the elongation of the elastic strand is retarded when the elastic strand has been stretched to approximately 140% of its original length.

9. The elastic strand according to claim 1, wherein the at least one outer strand includes between 15 and 30 outer strands.

10. The elastic strand according to claim 9, wherein the at least one outer strand includes between 20 and 25 outer strands.

11. A strip of kinesiology tape including the elastic strand of claim 1.

12. A strip of kinesiology tape, the strip of kinesiology tape comprising:
a fabric, wherein the fabric includes a weave of strands, wherein the strands include:
an elastic strand, wherein the elastic strand comprises:
an elastic core, wherein the elastic core is configured to:
elongate under an external force; and
return to its original size after an external force is removed; and
at least one outer strand, wherein the outer strand is wound around the elastic core;
a longitudinal cut in the fabric, wherein the longitudinal cut:
passes through at least a portion of the fabric; and
extends from a first edge of the fabric to a pre-determined distance from a second edge of the fabric, wherein the first edge is opposite the second edge; and
adhesive on a first surface of the fabric.

13. The strip of kinesiology tape according to claim 12, wherein the fabric is approximately rectangular in shape.

14. The strip of kinesiology tape according to claim 12, wherein the fabric includes a rounded exterior corner.

15. The strip of kinesiology tape according to claim 12, wherein the outer strand includes rayon.

16. A strip of kinesiology tape, the strip of kinesiology tape comprising:
a fabric, wherein the fabric includes a weave of strands, wherein the weave of strands includes:
a first set of strands, wherein each of the strands are oriented approximately parallel to one another; and
a second set of strands, wherein each of the strands in the second set of strands:
are oriented approximately parallel to one another;
are oriented approximately perpendicular to the first set of strands; and
includes an elastic strand, wherein the elastic strand comprises:
an elastic core, wherein the elastic core is configured to:
elongate under an external force; and
return to its original size after an external force is removed; and
at least one outer strand, wherein the outer strand is wound around the elastic core;
wherein the fabric:
is approximately rectangular in shape; and
includes rounded corners on all exterior corners;
a longitudinal cut in the fabric, wherein the longitudinal cut:
passes through at least a portion of the fabric;
extends from a first edge of the fabric to a pre-determined distance from a second edge of the fabric, wherein the first edge is opposite the second edge; and
is parallel to the second set of strands; and
adhesive on a first surface of the fabric, wherein the adhesive is applied in a step frequency wave pattern;
wherein the step-frequency wave pattern includes a sine wave pattern with modified peaks; and
wherein the adhesive is configured to adhere the fabric to a human body.

17. The strip of kinesiology tape according to claim 16, further comprising a backing material on the first surface of the fabric, wherein the backing material is configured to protect the adhesive until a user is ready to apply the fabric to the human body.

18. The strip of kinesiology tape according to claim 16, wherein the first set of strands include between 60 strands per inch and 66 strands per inch.

19. The strip of kinesiology tape according to claim 18, wherein the second set of strands include between 60 strands per inch and 66 strands per inch.

20. A roll of kinesiology tape, wherein the roll of kinesiology tape includes:
a first strip of kinesiology tape according to claim 16; and
a second strip of kinesiology tape according to claim 16.
